(19) United States
(12) Patent Application Publication
(10) Pub. No.: US 2020/0376229 A1
(43) Pub. Date: Dec. 3, 2020

(54) MIGRAINE TREATMENT PREPARATION AND METHOD

(71) Applicants: Bernie KASTNER, Jerusalem (IL); Gerard WERTMAN, Greensboro, CA (US)

(72) Inventors: Bernie KASTNER, Jerusalem (IL); Gerard WERTMAN, Greensboro, CA (US)

(21) Appl. No.: 16/934,032

(22) Filed: Jul. 21, 2020

Related U.S. Application Data
(63) Continuation-in-part of application No. PCT/US2018/056891, filed on Dec. 16, 2018.
(60) Provisional application No. 62/619,825, filed on Jan. 21, 2018.

(51) Int. Cl. A61M 21/00; A61M 31/00

(52) U.S. Cl. CPC A61M 21/00; A61M 2210/0643; A61M 2021/0077; A61M 31/00

(57) ABSTRACT

A method of ameliorating a migraine utilizing a frozen caffeinated comestible, the method constituted of: a) abutting a portion of the frozen caffeinated comestible to a user palate for at least 5 seconds; b) detaching the abutted portion from the user palate for a detachment period; and c) continuously repeating steps a-b until at least 70 milligrams of caffeine from the frozen caffeinated comestible have been consumed over a total period of at least 4 minutes.

### CROSS-REFERENCE TO RELATED APPLICATIONS

The current application is a continuation in part of PCT application PCT/US2018/065891, filed Dec. 16, 2018 and entitled “MIGRAINE TREATMENT PREPARATION”, the entire contents of which are incorporated by reference. PCT application PCT/US2018/065891 claims priority from U.S. provisional patent application Ser. No. 62/619,825, filed Jan. 21, 2018 and entitled “MIGRAINE TREATMENT PREPARATION”, the entire contents of which are incorporated herein by reference.

### TECHNICAL FIELD

The invention relates generally to the field of therapeutic preparations for migraine sufferers.

### BACKGROUND

Approximately 39 million people in the United States, representing approximately 12% of the population, are migraine sufferers. The frequency varies from individual to individual; some suffer a migraine 3-4 times a week while others suffer an episode once every two weeks. Symptoms range from nausea and vomiting to a severe pounding headache lasting several hours, with relief coming from resting in a dark room, lying down to sleep, and/or taking pain relief pills. The effects render the patient dysfunctional at work, and many hours of otherwise productive work are lost due to migraine's debilitating effect on the individual. Parents who suffer at home are not capable of taking care of their children and often leave them at risk of essentially being alone without parental supervision.

Numerous migraine medications exist; however they are expensive and often cause a myriad of side effects. For example, one FDA approved medication contains aspirin (acetylsalicylic acid), acetaminophen (also known as paracetamol) and caffeine (also known as Guaranine, Methyltheobromine, or 1,3,7-trimethylxanthine). Unfortunately, analgesics such as aspirin and acetaminophen do not work on the migraine process and can backfire, causing medication overuse headaches. Moreover, if the onset of a migraine comes on suddenly at a place of employment, the migraine sufferer is typically compelled to leave the place of employment due to inadequate conditions for treatment, i.e. not being able to lie down on a bed in a dark space.

Caffeine is known to be a migraine inhibitor for a good percentage of migraine sufferers. Before a headache or migraine, blood vessels tend to enlarge. Because it contains “vasoconstrictive” properties that cause the blood vessels to narrow and restrict blood flow, caffeine can aid in head pain relief. When caffeine is added to the combination of acetaminophen and aspirin, the pain relieving effect is increased by 40%.

Sphenopalatine ganglioneuralgia, commonly called brain freeze, is typified by a short-term headache lasting from a few seconds to a few minutes, resulting from ingesting a cold substance, such as ice cream or ice water. Sphenopalatine ganglioneuralgia is seemingly caused by the sphenopalatine ganglioneuralgia nerves (SPG), which is a group of nerves near the trigeminal nerve in the brain. While the precise mechanism is not yet clearly understood, it is believed that sphenopalatine ganglioneuralgia is a result of rapid dilation, and resultant increased blood flow, of either the arterial cerebral artery, or sinus capillaries, after constriction due to the rapid cooling of the affected blood vessels. Many migraine sufferers have reported that deliberately triggering an episode of brain freeze relieves the onset of a migraine.

What is desired, and is not yet available from the prior art, is a mechanism which combines the above mentioned remedies in a readily available form.

### SUMMARY OF THE INVENTION

Accordingly, it is a principal object of the present invention to overcome at least some of the disadvantages of the prior art. This is provided in one embodiment by providing a method of ameliorating a migraine utilizing a frozen caffeinated comestible, the method comprising: a) abutting a portion of the frozen caffeinated comestible to a user palate for at least 5 seconds; b) detaching the abutted portion from the user palate for a detachment period; and c) continuously repeating steps a-b until at least 70 milligrams of caffeine from the frozen caffeinated comestible has been consumed over a total period of at least 4 minutes.

In one embodiment, the abutting is to a mid-line of a soft palate/hard palate intersection. In another embodiment, the abutting is within a centimeter of a mid-line of a soft palate/hard palate intersection.

In one embodiment, the abutting to the user palate is to a point 1 centimeter posterior of a soft palate/hard palate intersection. In one further embodiment, the abutting to the point 1 centimeter posterior of the soft palate/hard palate intersection is at a mid-line of the soft palate/hard palate intersection. In another embodiment, the abutting is to the user palate at a mid-line of a line defined by the distal end of the patient left maxillary molar to the distal end of the patient right maxillary molar 65.

In one embodiment, steps a-b are continuously repeated until 120-200 milligrams of caffeine from the frozen caffeinated comestible has been consumed. In another embodiment, the total period is at least 5 minutes.

In one embodiment, the total period is at least 7 minutes. In another embodiment, during each iteration of step a), the abutted portion becomes liquid, and the subsequent iteration is to an adjacent portion of the frozen caffeinated comestible.

In one embodiment, the method further comprises separating an end portion of the frozen caffeinated comestible, the abutting performed on the separated end portion. In another embodiment, the frozen caffeinated comestible has an initial volume of 100-150 milliliters.

In one embodiment, the frozen caffeinated comestible has an initial volume of about 125 milliliters. In another embodiment, the frozen caffeinated comestible exhibits a concentration of at least 0.7 mg caffeine/milliliter.

In one embodiment, the frozen caffeinated comestible is configured as an elongate device having a length at least 4 times a width thereof. In another embodiment, the frozen caffeinated comestible is an aqueous solution.

In one embodiment, the frozen caffeinated comestible comprises a sweetener. In another embodiment, the frozen caffeinated comestible comprises a plurality of separated sections, wherein the abutting comprises abutting each of the plurality of separated sections subsequent to a previously abutted section melting.

In one embodiment, step b) is performed for no more than 30 seconds.

In one independent embodiment, a frozen comestible for treatment of migraine onset is enabled, comprising at least 70 mg caffeine and exhibiting a first end and a second end, opposing the first end, wherein a face of the first end is configured and dimensioned to fit over a palatine raphe.

In one embodiment, a face of the first end exhibits a depression. In another embodiment, the first end is generally m-shaped.

In one embodiment, the frozen comestible comprises 120-200 milligrams of caffeine. In one further embodiment, the frozen comestible comprises about 200 milligrams of caffeine.

In another embodiment, the frozen comestible has a volume of 100-150 milliliters. In one further embodiment, the frozen comestible has a volume of about 125 milliliters.

In one embodiment, the frozen comestible exhibits a concentration of at least 0.7 mg caffeine/milliliter. In another embodiment, the frozen comestible is configured as an elongate device having a length at least 4 times a width thereof.

In one embodiment, the frozen comestible is constituted as an aqueous solution. In another embodiment, the frozen comestible further comprises a sweetener.

In one embodiment, the frozen comestible is separated into a plurality of sections.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding sections or elements throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice. In the accompanying drawings:

FIG. 1A illustrates a high level schematic diagram of a frozen caffeinated comestible, in accordance with certain embodiments;

FIG. 1B illustrates a high level schematic diagram of a palate;

FIG. 1C illustrates a high level schematic diagram of the frozen caffeinated comestible being applied to the palate of FIG. 1B, in accordance with certain embodiments;

FIG. 1D illustrates a high level side view of a palate shaped end of the frozen caffeinated comestible;

FIG. 1E illustrates a high level flow chart of a method of ameliorating a migraine utilizing the frozen caffeinated comestible of FIG. 1A;

FIG. 2A illustrates a high level schematic diagram of a frozen caffeinated comestible, separated into a plurality of sections, in accordance with certain embodiments;

FIG. 2B illustrates a high level schematic diagram of the frozen caffeinated comestible of FIG. 2A stored in a package comprising a plurality of compartments, in accordance with certain embodiments;

FIG. 2C illustrates a high level schematic diagram of the frozen caffeinated comestible of FIG. 2A stored in a plurality of packages, in accordance with certain embodiments; and

FIG. 2D illustrates a high level flow chart of a method of ameliorating a migraine utilizing the frozen caffeinated comestible of FIG. 2A.

### DETAILED DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

The device and method as described herein has been shown to be helpful in alleviating migraine symptoms. Advantageously, the device is readily obtainable, and can be stored in frozen form for long periods of time, and easily transported in the frozen form in a user insulated bag, so as to be readily available for immediate use upon the onset of symptoms. The device combines the advantages of the prior art in an easily accessible device. Particular application instructions of the method provide for an improved response which is not provided by the various known home remedies.

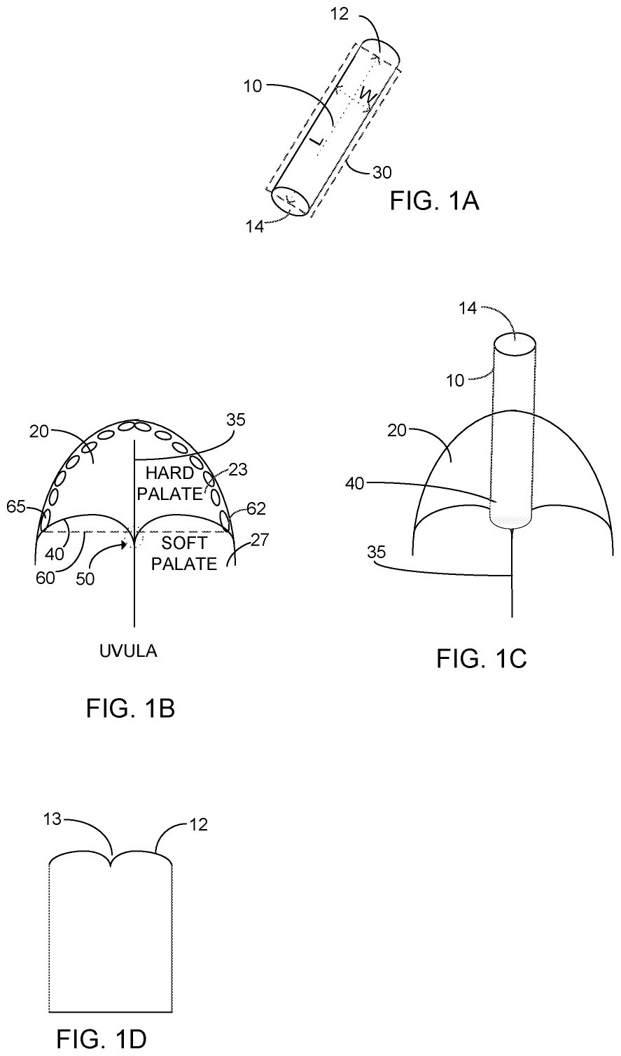

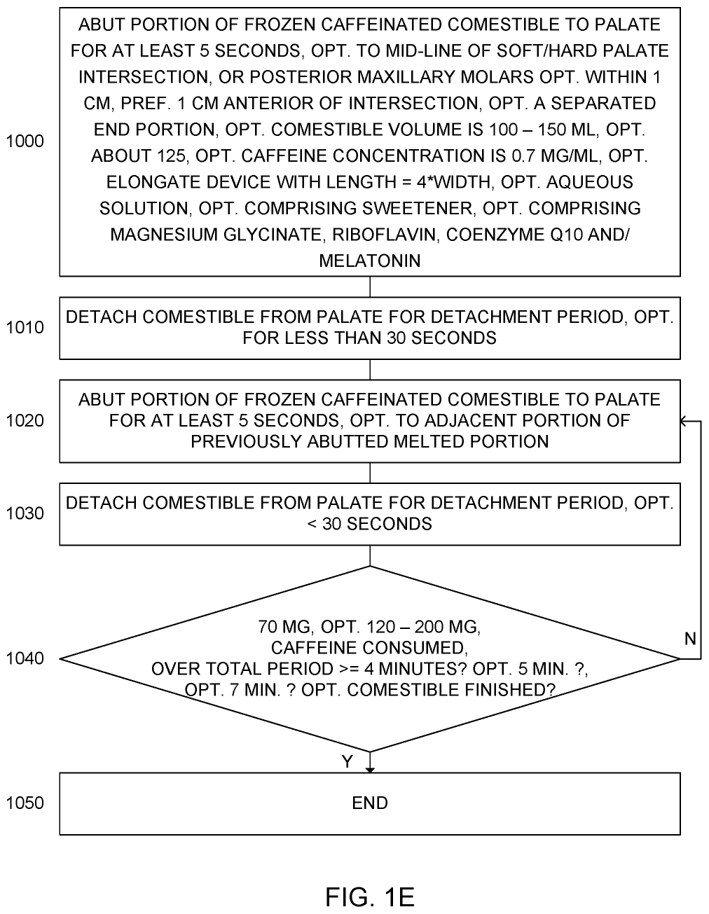

FIG. 1A illustrates a high level schematic diagram of a frozen caffeinated comestible 10. FIG. 1B illustrates a high level schematic diagram of a palate 20. FIG. 1C illustrates a high level schematic diagram of frozen caffeinated comestible 10 being applied to the palate of FIG. 1B, in accordance with certain embodiments. FIG. 1D illustrates a high level schematic diagram a palate shaped end of frozen caffeinated comestible 10. FIG. 1E illustrates a high level flow chart of a method of ameliorating a migraine utilizing frozen caffeinated comestible 10, FIGS. 1A-1E being described together.

For brevity, frozen caffeinated comestible 10 is described herein as ‘comestible’ 10. The term ‘comestible’, as used herein, is meant as an edible item. The term ‘caffeinated’, as used herein, means that comestible 10 contains caffeine. Particularly, comestible 10 comprises at least 70 milligrams of caffeine and a sweetener. In one embodiment, comestible 10 comprises at least 120 milligrams of caffeine. In one further embodiment, comestible 10 comprises 120-150 milligrams of caffeine. In another embodiment, comestible 10 comprises more than 150 milligrams of caffeine. In one further embodiment, comestible 10 comprises 150-200 milligrams of caffeine. In one yet further embodiment, comestible 10 comprises about 200 milligrams of caffeine. Advantageously, in the embodiment where comestible 10 comprises more than 150 milligrams of caffeine, the user will have consumed more than 70 milligrams of caffeine even if less than half of comestible 10 is consumed, as will be described below. Increasing the amount of caffeine to 200 milligrams of caffeine will ensure that the user consumes at least 100 milligrams of caffeine, even if only half of comestible 10 is consumed. Standard non-prescription caffeine pills generally have 200 milligrams of caffeine, which indicates that such an amount of caffeine is considered safe. The term ‘frozen’, as used herein, means that comestible 10 is cooled to a solid state, as will be described below.

In one embodiment, comestible 10 additionally comprises Magnesium Glycinate, Riboflavin, Coenzyme Q10 and/or Melatonin. In one further embodiment, comestible 10 comprises about 5 milligrams of Magnesium Glycinate, about 10 milligrams of Riboflavin, about 5 milligrams of Coenzyme Q10 and/or about 200 micrograms of Melatonin. Adding one or more of these ingredients increases the analgesic effect of comestible 10 for the alleviation of migraine symptoms.

Comestible 10 is provided inside a package 30. In one embodiment, comestible 10 has a volume of 100-150 milliliters. In one further embodiment, comestible 10 has a volume of about 125 milliliters. In another embodiment, the concentration of caffeine in comestible 10 is at least 0.7 milligrams of caffeine per milliliter. Preferably, the concentration of caffeine in comestible 10 is at least 1.0 milligrams of caffeine per milliliter.

Comestible 10 is formed of liquid which can be frozen, i.e. cooled to a solid state. In one embodiment, comestible 10 is provided in package 30 in a liquid state and is frozen by a user. In another embodiment, comestible 10 is provided in package 30 in a frozen form. In one embodiment, comestible 10 is an aqueous solution. In another embodiment, comestible 10 comprises a sweetener.

In one embodiment, comestible 10 is produced by mixing water, caffeine and a sweetener. The solution is then frozen. In one further embodiment, liquid caffeine is used for creating comestible 10. In another embodiment, the sweetener is one or more of: glucose; sucrose, including from sugar beet or sugar cane; fructose; agave nectar; date sugar; honey; maple syrup; molasses; guarana; sorbitol; xylitol; stevia; tagatose; trehalose aspartame; sucralose; neotame; acesulfame potassium; saccharin; erythritol; hydrogenated starch hydrolysate; isomalt; lactitol; maltitol; mannitol; sorbitol; xylitole; advantame; cyclamate; allulose; and a mogroside, without limitation. It is noted that the above list is in no way limiting. In one embodiment, additional flavoring is also added. In another embodiment, natural and artificial sweeteners and flavorings can be mixed, such as, but not limited to: tea; coffee; and guarana.

In another embodiment, comestible 10 is produced by mixing fruit and/or vegetable juice and caffeine. In such an embodiment, the sweetener is provided naturally by the fruit juice. In one further embodiment, an additional sweetener is added. In another further embodiment, additional water is added. In one embodiment, the juice is derived from one or more of: apples; apricots; bananas; figs; grapes; oranges; peaches; pears; pineapples; plums; beets; taro; carrots; corn; peppers; onions; sweet potatoes; and yams. It is noted that the above list is in no way limiting.

In another embodiment, comestible 10 is produced by mixing milk, caffeine and a sweetener. In one further embodiment, one or more additional dairy products, such as cream, is added.

Comestible 10 exhibits a first end 12 and a second end 14, second end 14 opposing first end 12. In one embodiment, comestible 10 is elongated, extending from first end 12 to second end 14. In one further embodiment, a length of comestible 10, denoted L, from first end 12 to second end 14, is at least 4 times a width of comestible 10, the width denoted as W. Particularly, in a comestible 10 with 3 different dimension sizes, the largest dimension is termed herein the length, L; the smallest dimension is termed herein the depth; and the dimension larger than, or equal to, the depth, but smaller than the length, is termed herein the width, W. In one embodiment, width W of comestible 10 is 2.5-5.5 centimeters. In one further embodiment, width W of comestible 10 is about 3.2 centimeters, which is the average width of a palate. In one embodiment, comestible 10 is configured and dimensioned within package 30 so as to generally form a cylindrical shape when frozen. In another embodiment, as illustrated in FIG. 1D, first end 12 is shaped and configured to fit palate 20. Particularly, in one embodiment, the face of first end 12 exhibits a depression 13, depression 13 shaped and configured to fit over the palatine raphe. In one embodiment, first end 12 is m-shaped, i.e. shaped like the letter ‘m’. In another embodiment, depression 13 is generally in the middle of first end 12. First end 12 of comestible 10, when frozen, is applied to a midline 35 of a user soft palate/hard palate intersection 40, as shown by the target area 50, as will be described below. Midline 35 is defined by the user palatine raphe. In the embodiment, where first end 12 comprises depression 13, depression 13 allows increased contact of first end 12 with palate 20 without being blocked by the palatine raphe.

Particularly, in stage 1000, a user suffering from onset of a migraine abuts first end 12 of comestible 10 to palate 20 at area 50. In a first embodiment, first end 12 is abutted to midline 35 of the soft palate/hard palate intersection 40 of palate 20. Specifically, soft palate/hard palate intersection 40 is defined herein where the hard palate 23 and soft palate 27 meet and midline 35 is defined as the palatine raphe, which is a raphe running from the palatine uvula to the incisive papilla, and thus crosses soft palate/hard palate intersection 40 defining area 50. In another embodiment, area 50 is defined as a 1 centimeter centered on the intersection of midline 35 and soft palate/hard palate intersection 40. In one preferred embodiment area 50 is defined as a 1 centimeter posterior of soft palate/hard palate intersection 40, preferably centered on the intersection of midline 35. In yet another embodiment, first end 12 is abutted to an area of palate 20 at an area 50 defined by the intersection of: a line 60 drawn from the distal end of the patient left maxillary molar 62 to the distal end of the patient right maxillary molar 65, with distal defined as furthest from the patient incisive papilla; and midline 35. In one embodiment the patient is instructed to abut first end 12 to the intersection of midline 35 with the most posterior one of line 60 and the posterior end of the hard palate 23, and not further than the start of the soft palate. This area is where the second branch of the trigeminal nerve sits. The low temperature of the comestible 10 contacting palate 20, along with the caffeine within comestible 10, relieves the onset of the migraine. Particularly, due to the abutment of comestible 10 with palate 20, the caffeine from comestible 10 is absorbed directly through palate 20 into the second branch of the trigeminal nerve.

It is to be noted that the particular placement of comestible 10 to contact palate 20 is not normally accomplished in the consuming of a popsicle or other frozen treat. Furthermore, the preferred area 50, as described above, is significantly more posterior than is normally accessed by frozen treats.

First end 12 is preferably abutted to palate 20, preferably at area 50, for a period at least 5 seconds, and for as long as it is tolerated. Preferably, first end 12 is maintained in contact with palate 20 for as long as it is tolerated, but for no less than 5 seconds to ensure effectiveness. In one embodiment, first end 12 of comestible 10 is abutted with palate 20 by pushing the entirety of comestible 10 towards the roof of the mouth. In another embodiment, the user bites off a section of first end 12 with the user incisors, or cuts of a lead section with a knife, and holds the section against palate 20 with their tongue for the period of at least 5 seconds, and for as long as it is tolerated.

In stage 1010, first end 12 of comestible 10, or the portion thereof, is detached from palate 20 for a detachment period. During the detachment period, palate 20 warms up to recover from the uncomfortable cold impact. Particularly, the detachment period is as short as possible, until the user feels that they are able to again abut comestible 10 to palate 20. Preferably, the detachment period is no more than 30 seconds.

In stage 1020, comestible 10 of stage 1010, or a portion thereof, is again abutted to palate 20 for a period of at least 5 seconds, as described above in relation to stage 1000. Due to the fact that in stage 1000 first end 12 melted into liquid from the contact with palate 20, a portion of comestible 10 adjacent to first end 12, which has not yet melted, is abutted to palate 20. In the embodiment wherein sections are being handled individually either by biting, or cutting, the following section of comestible 10, which now defined first end 12, is now cut.

In stage 1030, comestible 10 of stage 1020 is detached from palate 20, as described above in relation to stage 1010. In stage 1040, stages 1020-1030 are repeated until at least 70 milligrams of caffeine have been consumed, over a total period of no less than 4 minutes. The term ‘consumed’, as used herein, means that a first portion of the caffeine is absorbed through palate 20 and a second portion of the caffeine is swallowed. Since comestible 10 is in contact with palate 20, the portion of absorbed caffeine should be significantly greater than the portion of swallowed caffeine, which increases the effectivity of the treatment. As described above, comestible 10 comprises at least 70 milligrams of caffeine, thereby at least 70 milligrams of caffeine is consumed if the entirety of comestible 10 is consumed. As further described above, at least 70 milligrams of caffeine will be consumed even when not all of comestible 10 has been consumed in the embodiment where comestible 10 comprises a greater amount of caffeine.

In one preferred embodiment, stages 1020-1030 the total period is no less 5 minutes. In one further embodiment, stages 1020-1030 the total period is no less than 7 minutes. This provides application of cold temperature to the trigeminal nerve for a sufficient amount of time to ameliorate the migraine. Alternatively, stages 1020-1030 are repeated until comestible 10 has been completely consumed, regardless of the time period. It is noted that at a certain point, the length of comestible 10 may be reduced by the melting such that second end 14 is no longer external to the user's mouth.

The inventor conducted a trial for testing the efficacy of the above described treatment. Volunteers diagnosed by a physician as suffering from migraines were given a frozen caffeinated comestible 10 and instructed to abut first end 12 of frozen caffeinated comestible 10 to palate 20, as described above, upon the onset of a migraine attack. 80% of the users reported some level of symptom relief. Additionally, 60% of those who reported some level of relief reported that there was no need to take any further medication for the relief of migraine symptoms experienced during that migraine attack.

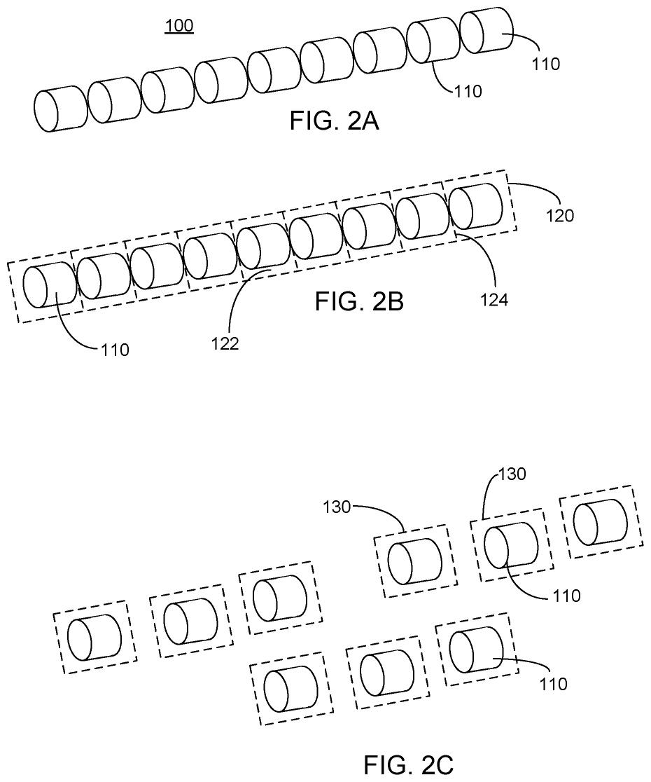

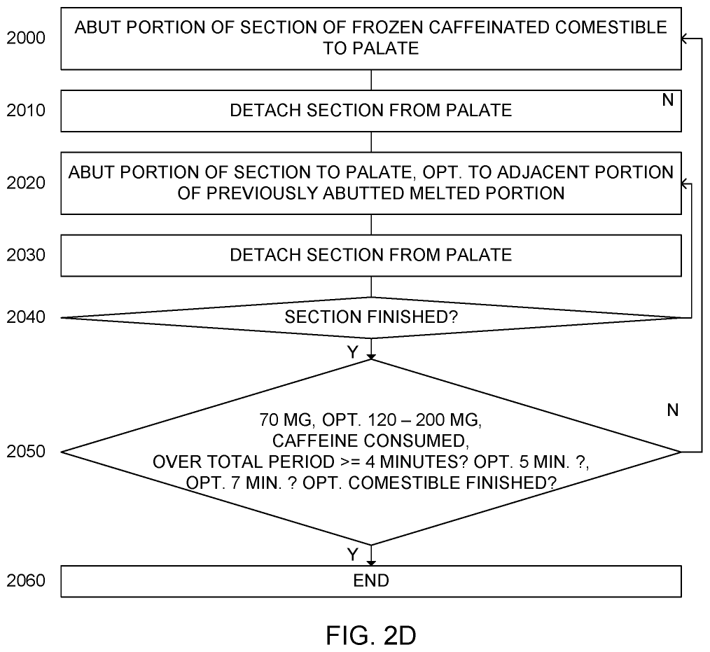

Although the above has been described in relation to a comestible 10 comprising a single frozen piece, this is not meant to be limiting in any way. In another embodiment, illustrated in FIG. 2A, a frozen caffeinated comestible 100 is provided, frozen caffeinated comestible 100 described herein as comestible 100. Comestible 100 is in all respects similar to comestible 10 with the exception that comestible 100 comprises a plurality of separated sections 110. Particularly, in one embodiment, as illustrated in FIG. 2B, comestible 100 is provided within a package 120, package 120 comprising a plurality of compartments 122. Compartments 122 are separated from each other by respective walls 124 and each compartment 122 contains a respective section 110. In another embodiment, as illustrated in FIG. 2C, each section 110 of comestible 100 is provided in a respective one of a plurality of packages 130. In one embodiment, each section 110 exhibits a face with a width of 2-3 centimeters and a length of about 3.2 centimeters. As described above, in one embodiment each section is palate shaped. Although 9 sections 110 are illustrated in each of FIGS. 2A-2C, this is not meant to be limiting in any way. FIG. 2D illustrates a high level flow chart of a method of ameliorating a migraine utilizing comestible 100.

In stage 2000, a portion of a section 110 of comestible 100 is abutted to a user palate, as described above in relation to stage 1000. As described above, section 110 is abutted to the palate for at least 5 seconds, preferably for as long as possible. In one embodiment (not shown), a holding device is provided which is configured and dimensioned to contain section 110. The user inserts the holding device into their mouth and use the holding device to perform the above described abutment. In stage 2010, section 110 of stage 2000 is detached from the palate for a detachment period, as described above in relation to stage 1010. As described above, the detachment period is preferably as short as possible, until the user is able to again abut section 110 to the palate. Further preferably, the detachment period is less than 30 seconds.

In stage 2020, section 110 of stage 2010, is again abutted to the palate, as described above in relation to stage 2000. Due to the fact that in stage 2000 a portion of section 110 melted into liquid from the contact with the palate, an adjacent portion, which has not yet melted, is abutted to the palate. In stage 2030, section 110 of stage 2020 is detached from the palate for a detachment period, as described above in relation to stage 2020. In stage 2040, stages 2020-2030 are repeated until section 110 is finished, i.e. has been completely consumed. Although the above has been described where at least two abutment iterations are performed, this is not meant to be limiting in any way, and section 110 may be consumed without detachment from the palate.

In stage 2050, stages 2000-2040 are repeated utilizing another section 110 of comestible 100 until at least 70 milligrams of caffeine have been consumed, over a total period of no less than 4 minutes, as described above in relation to stage 1040. Preferably, stages 2000-2040 are repeated until all sections 110 are consumed.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and subcombinations of the various features described hereinabove as well as variations and modifications thereof which would occur to persons skilled in the art upon reading the foregoing description.

1. A method of ameliorating a migraine utilizing a frozen caffeinated comestible, the method comprising:
a) abutting a portion of the frozen caffeinated comestible to a user palate for a period of at least 5 seconds;
b) detaching said abutted portion from the user palate for a detachment period; and
c) continuously repeating steps a-b until at least 70 milligrams of caffeine from the frozen caffeinated comestible has been consumed over a total period of at least 4 minutes.

2. The method of claim 1, wherein said abutting is to a mid-line of a soft palate/hard palate intersection.

3. The method of claim 1, wherein said abutting is within a centimeter of a mid-line of a soft palate/hard palate intersection.

4. The method of claim 1, wherein said abutting to the user palate is to a point 1 centimeter posterior of a soft palate/hard palate intersection.

5. The method of claim 1, wherein said abutting is to the user palate at a mid-line of a line defined by the distal end of the patient left maxillary molar to the distal end of the patient right maxillary molar 65.

6. The method of claim 1, wherein steps a-b are continuously repeated until 120-200 milligrams of caffeine from the frozen caffeinated comestible has been consumed.

7. The method of claim 1, wherein the total period is at least 5 minutes.

8. The method of claim 1, wherein during each iteration of step a), the abutted portion becomes liquid, and the subsequent iteration is to an adjacent portion of the frozen caffeinated comestible.

9. The method of claim 1, wherein the frozen caffeinated comestible exhibits a concentration of at least 0.7 mg caffeine/milliliter.

10. The method of claim 1, wherein the frozen caffeinated comestible further comprises: Magnesium Glycinate; Riboflavin; Coenzyme Q10; and Melatonin.

11. The method of claim 1, wherein the frozen caffeinated comestible is configured as an elongate device having a length at least 4 times a width thereof.

12. The method of claim 1, wherein the frozen caffeinated comestible comprises a plurality of separated sections, wherein said abutting comprises abutting each of said plurality of separated sections subsequent to a previously abutted section melting.

13. The method of claim 1, wherein step b) is performed for no more than 30 seconds.

14. A frozen comestible for treatment of migraine onset comprising at least 70 mg caffeine and exhibiting a first end and a second end, opposing said first end, wherein a face of said first end is configured and dimensioned to fit over a palatine raphe.

15. The frozen comestible of claim 14, wherein a face of said first end exhibits a depression.

16. The frozen comestible of claim 14, wherein said first end is generally m-shaped.

17. The frozen comestible of claim 14, comprising 120-200 milligrams of caffeine.

18. The frozen comestible of claim 14, exhibiting a concentration of at least 0.7 mg caffeine/milliliter.

19. The frozen comestible of claim 14, further comprising: Magnesium Glycinate; Riboflavin; Coenzyme Q10; and Melatonin.

20. The frozen comestible of claim 14 configured as an elongate device having a length at least 4 times a width thereof.
